(19) United States
(12) Patent Application Publication
(10) Pub. No.: US 2026/0260765 A1
(43) Pub. Date: Sep. 3, 2026

(54) APPARATUS AND METHOD OF GENERATING DATA FABRIC-BASED DISEASE-SPECIFIC DATABASE

(71) Applicant: KaKao Healthcare Corp., Seongnam-si (KR)

(72) Inventors: Yonghyun CHO, Seongnam-si (KR); Dahye SHIN, Seongnam-si (KR)

(21) Appl. No.: 18/873,460

(22) PCT Filed: Feb. 14, 2024
(86) PCT No.: PCT/KR2024/095113

§ 371 Date: Dec. 10, 2024

(30) Foreign Application Priority Data
Feb. 14, 2023 (KR) 10-2023-0019308
Feb. 13, 2024 (KR) 10-2024-0020558

(51) Int. Cl. G16H 50/70; G06F 3/0482

(52) U.S. Cl. CPC G16H 50/70; G06F 3/0482

(57) ABSTRACT

Disclosed is a method of an apparatus operating by at least one processor, the method comprising: constructing a medical data library that includes data items extractable from a clinical data warehouse for disease-specific study, and generates a table of each data item by applying a disease-specific fabric condition that includes medical conditions for extracting a disease-specific patient group; obtaining a request to generate a database for study of a specific disease; determining, from the medical data library, specific data items relevant to the study of the specific disease; generating a table of each of the specific data items using data for a patient group of the specific disease defined by applying a fabric condition of the specific disease; and generating a database for the study of the specific disease using the tables of the specific data items.

### TECHNICAL FIELD

The present disclosure relates to a medical data library.

### BACKGROUND ART

A Clinical Data Warehouse (CDW) contains vast amounts of data from a Hospital Information System (HIS), an Electronic Medical Record (EMR), an Order Communication System (OCS), and the like. Therefore, researchers may extract the desired medical data from the CDW to conduct medical studies.

Real-World Evidence (RWE) is clinical evidence derived by processing and analyzing Real-World Data (RWD), which is variously generated and collected in the real environment. RWE has the potential to address the ethical concerns associated with Randomized Controlled Trials (RCTs) that involve patient recruitment, significantly reduce time and cost, and is broadly accessible.

For RWE analysis, researchers must build a database for study purpose by designing a research hypothesis, extracting a patient group that meets the criteria from the CDW, and continuously adding the required data. This approach can be time-consuming to build the database and may result in missing critical data. In addition, the existing study-specific database cannot be reused for other studies, requiring data extraction to be repeated for each new study purpose.

### DISCLOSURE

### Technical Problem

The present disclosure attempts to provide an apparatus and a method of generating a data fabric-based disease-specific database.

The present disclosure also attempts to provide an apparatus and a method of generating a disease-specific database by building a medical data library from which data items are selectable based on a data fabric, and combining the selected items for the study of a specific disease.

### Technical Solution

An embodiment of the present disclosure provides a method of an apparatus operating by at least one processor, the method comprising: constructing a medical data library that includes data items extractable from a clinical data warehouse for disease-specific study, and generates a table of each data item by applying a disease-specific fabric condition that includes medical conditions for extracting a disease-specific patient group; obtaining a request to generate a database for study of a specific disease; determining, from the medical data library, specific data items relevant to the study of the specific disease; generating a table of each of the specific data items using data for a patient group of the specific disease defined by applying a fabric condition of the specific disease; and generating a database for the study of the specific disease using the tables of the specific data items.

The determining the specific data items may comprise: providing a user interface with selectable data items for the study of the specific disease; and determining data items confirmed in the user interface to the specific data items.

The providing the user interface may comprise managing default items selected from the medical data library by disease or study purpose, and providing the user interface with default items related to the request to generate the database.

The generating the table of each of the specific data items may comprise, when a table of a specific data item needs to be generated from unstructured data, extracting a table record value from the unstructured data using an artificial intelligence model to generate the table of the specific data item.

The generating the table of each of the specific data items may comprise: determining an order of table generation of the specific data items; and generating the table according to the order.

The data items in the medical data library may be classified into any one of a core part, an extension part, or an analysis part. The core part may include data items related to essential clinical information being used as a default for studies. The extension part may include data items related to clinical information being optionally used based on a disease or study purpose. And the analysis part may include data items related to derivative information necessary for studying and analyzing clinical information.

The obtaining the request to generate the database may comprises receiving an input of a disease name of the specific disease.

The method may further comprise: after obtaining the request to generate the database, extracting a number of patients with the specific disease from the clinical data warehouse and providing the number of patients of the specific disease.

Another embodiment of the present disclosure provides a method of an apparatus operating by at least one processor, the method comprising: obtaining a table specification of target item to be added in a medical data library; classifying the target item into any one of a core part, an extension part, or an analysis part of the medical data library based on a classification criterion; and generating table information and structure of the target item into a structured query language code, and registering the target item in the medical data library. The medical data library is constructed to include data items extractable from a clinical data warehouse for disease-specific study, and to generate a table of each data item by applying a disease-specific fabric condition that includes medical conditions for extracting a disease-specific patient group.

The classifying may comprise: when the target item is essential clinical information being used as a default for studies, classifying the target item into the core part; when the target item is clinical information being optionally used for a disease or study purposes, classifying the target item into the extension part; and when the target item is derivative information necessary for studying and analyzing clinical information, classifying the target item into the analysis part.

The method may further comprise setting an order of table generation based on table information of the target item.

The method may further comprise: when a table for the target item needs to be generated from unstructured data, adding an artificial intelligence model to a table generation process for the target item to convert the unstructured data into a structured table.

The method may further comprise: obtaining a request to generate a database for study of a specific disease; determining, from the medical data library, specific data items relevant to the study of the specific disease; generating a table of each of the specific data items using data for a patient group of the specific disease defined by applying a fabric condition of the specific disease; and generating a database for the study of the specific disease using the tables of the specific data items.

Still another embodiment of the present disclosure provides an apparatus comprising: a memory; and a processor for executing instructions stored in the memory. The processor is configured to, by executing the instructions, construct a medical data library to include data items extractable from a clinical data warehouse for disease-specific study, and to generate a table of each data item by applying a disease-specific fabric condition that includes medical conditions for extracting a disease-specific patient group, generate a table of each of the data items selected in the medical data library using data for a patient group of a specific disease defined by applying a fabric condition of the specific disease, and provide a database including the generated tables for study of the specific disease.

The processor may be configured to obtain a request to generate a database for study of the specific disease, determine, from the medical data library, specific data items relevant to the study of the specific disease, generate a table of each of the specific data items using data for a patient group of the specific disease defined by applying a fabric condition of the specific disease, and generate a database for the study of the specific disease using the tables of the specific data items.

The processor may be configured to provide a user interface with selectable data items in the medical data library for the study of the specific disease, and determine data items confirmed in the user interface to the specific data items.

The processor may be configured to manage default items selected from the medical data library by disease or study purpose, and provide the user interface with default items related to the request to generate the database.

The processor may be configured to, when a table of a specific data item needs to be generated from unstructured data, extract a table record value from the unstructured data using an artificial intelligence model to generate the table of the specific data item.

The processor may be configured to determine an order of table generation of the specific data items, and generate the table according to the order.

The data items in the medical data library may be classified into any one of a core part, an extension part, or an analysis part. The core part may include data items related to essential clinical information being used as a default for studies. The extension part may include data items related to clinical information being optionally used based on a disease or study purpose. And the analysis part may include data items related to derivative information necessary for studying and analyzing clinical information.

### Advantageous Effects

According to the embodiments, databases for real-world evidence (RWE) analysis may be configured quickly and sophisticatedly.

According to the embodiments, study-specific databases that rely on researcher's ability to be built may be generated with consistent quality.

According to the embodiments, the data items within the medical data library may be freely combined to quickly generate disease-specific databases for study purposes.

According to the embodiments, data items from the medical data library may be used by multiple researchers to generate customized databases, and previously generated disease-specific databases may be reused.

### DESCRIPTION OF DRAWINGS

FIG. 1 is a diagram illustrating an apparatus for providing a data fabric-based disease-specific database according to an embodiment.

FIG. 2 is a diagram illustrating a method of generating a table of artificial intelligence-based unstructured data according to the embodiment.

FIG. 3 is a diagram illustrating functions implemented in the apparatus for providing the data fabric-based disease-specific database according to the embodiment.

FIG. 4 is a diagram illustrating an example of generation of a disease-specific database according to the embodiment.

FIG. 5 is a flow diagram illustrating a method of building a medical data library according to an embodiment.

FIG. 6 is a flow diagram illustrating a method of generating a disease-specific database according to the embodiment.

### MODE FOR INVENTION

The present invention will be described more fully hereinafter with reference to the accompanying drawings, in which exemplary embodiments of the invention are shown. As those skilled in the art would realize, the described embodiments may be modified in various different ways, all without departing from the spirit or scope of the present invention. Accordingly, the drawings and description are to be regarded as illustrative in nature and not restrictive. Like reference numerals designate like elements throughout the specification.

Throughout the specification, unless explicitly described to the contrary, the word “comprise”, and variations such as “comprises” or “comprising”, will be understood to imply the inclusion of stated elements but not the exclusion of any other elements. In addition, the terms “-er”, “-or”, and “module” described in the specification mean units for processing at least one function and operation, and may be implemented by hardware components or software components, and combinations thereof.

The device may include one or more processors, a memory for loading computer programs executed by the processors, a storage device for storing the computer programs and various data, and a communication interface. In addition, the device may further include various components

A processor is a device that controls the operation of a device and may be various forms of processor that processes instructions contained in a computer program, and may include, for example, at least one of a Central Processing Unit (CPU), a Micro Processor Unit (MPU), a Micro Controller Unit (MCU), a Graphic Processing Unit (GPU), or any other form of processor well known in the art of the present disclosure. The memory stores various data, instructions, and/or information. The memory may load a corresponding computer program from the storage device such that the instructions described to execute the operations of the present disclosure are processed by the processor. The memory may be, for example, Read Only Memory (ROM) and Random Access memory (RAM). The storage device may non-temporarily store computer programs and various data. The storage device may include a non-volatile memory, such as a Read Only Memory (ROM), an Erasable Programmable ROM (EPROM), an Electrically Erasable Programmable ROM (EEPROM), a flash memory, or the like, a hard disk, a removable disk, or any other form of computer-readable recording medium well known in the art to which the present disclosure belongs. The communication interface may be a wired/wireless communication module that supports wired/wireless communication. A computer program may include instructions executed by the processor, and may be stored on a non-transitory computer readable storage medium, and the instructions cause the processor to execute the operation of the present disclosure.

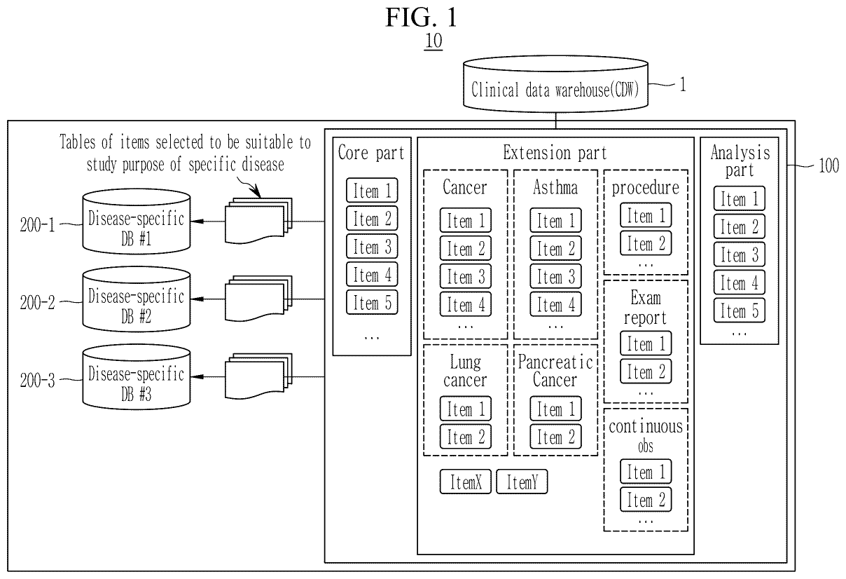

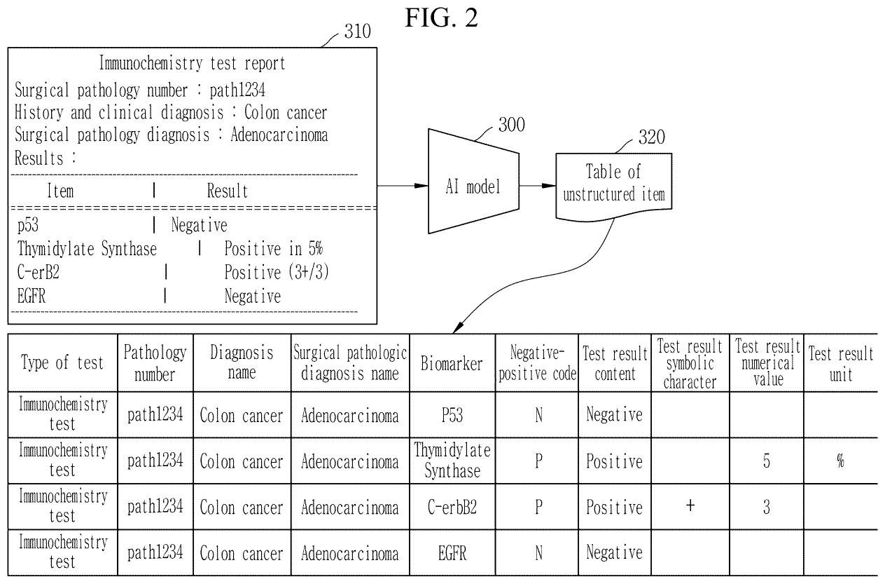

FIG. 1 is a diagram illustrating an apparatus for providing a data fabric-based disease-specific database according to an embodiment, and FIG. 2 is a diagram illustrating a method of generating a table of artificial intelligence-based unstructured data according to the embodiment.

Referring to FIG. 1, an apparatus 10 operated by at least one processor builds a medical data library 100 from which data items (simply referred to as “items”) may be selected based on a data fabric, and generates disease-specific databases (DBs) 200 (e.g., 200-1, 200-2, 200-3, . . .) by combining data from selected items for the study of a specific disease.

The apparatus 10 designs a table specification of each item to be extracted from a Clinical Data Warehouse (CDW) 1 and generates a code for generating a table with the specified information and structure. The information and structure of the table may be generated using a structured query language (e.g., SQL). The apparatus 10 stores fabric conditions that include medical conditions (criteria) for extracting patient groups by disease, and variables that need to be configured for the study. For example, the fabric conditions for lung cancer disease may include conditions for selecting patients diagnosed with both C33 and C34 subcodes of ICD-10, which are lung cancer disease classification codes, patients diagnosed in an oncology department or cancer hospital, and patients who had a chest X-ray or chest Computed Tomography (CT) scan performed before the first diagnosis date. The disease-specific fabric condition may be variously configured according to disease-specific clinical guidelines.

The apparatus 10 then generates tables of the items selected from the medical data library 100 in response to a request to generate a DB for study purposes of a specific disease. The apparatus 10 may apply a fabric condition for the specific disease to generate a table of each item, using only data for the patient group associated with the specific disease. The apparatus 10 may transform the medical data based on the fabric condition to generate a table of each selected item. The codes for transforming the medical data into a table based on the fabric condition may be generated in a structured query language (e.g., SQL). Here, for items used to generate the DB for study purposes of the specific disease, the apparatus 10 may default to selecting at least some of the items based on the specific disease and study purposes, may recommend selectable items to the user, and may allow the user to choose desired items.

The apparatus 10 may generate a DB for study purposes of a specific disease including the tables generated with the applied the fabric condition, and provide the completed DB to the requestor.

For example, when the apparatus 10 generates a database for “Study: Treatment Patterns Among Pancreatic Cancer Patients in the Intensive Care Unit”, when generating a table for each selected item, the apparatus 10 may apply a fabric condition that extracts a patient group who are treated by “admission to an intensive care unit,” and generate a table for each item using only data from the patient group with pancreatic cancer who have been admitted and treated in an intensive care unit.

For example, when the apparatus 10 generates a database for a “Study: Survival Analysis of Patients with Asthma and Lung Cancer”, when generating a table of each selected item, the apparatus 10 may apply a fabric condition that extracts a patient group related to “asthma” and “lung cancer”, and generate a table of each item using only data from the patient group with “asthma” and “lung cancer”.

The medical data library 100 may comprise selectable items by specific diseases and study purposes, and the items may be classified into a core part, an extension part, and an analysis part. New items may be added to the medical data library 100, allowing subsequent users to reuse the added items. The data of each item is transformed into a table with a defined information and structure. Each item may include further selectable detailed items, and the selected detailed items may form the columns of the table.

The core part may include items that contain essential clinical information being used as a default for the study analysis. Items classified into the core part may include information generated during a patient's hospital visit for care and treatment, which is common across all patients. For example, items classified into the core part may include patient information, demographics, encounter history, encounter, diagnoses, medication order, surgeries/procedures, diagnostic test results, and imaging test results. Patient information items may consist of detailed items, such as age, gender, nationality, blood type, and cause of death.

The extension part may include items that contain clinical information being optionally used based on the disease, applied condition, or study purpose. For example, items classified into the extension part may be classified and managed into categories, such as disease, procedure, test result, continuous observation, and other, and each category may be further subdivided. For example, a cancer disease may further include clinical items specific to a cancer type, such as lung cancer or pancreatic cancer. For example, cancer-related items may include cancer registration basics, cancer registration tumors, chemotherapy protocols, anticancer flowsheets, and radiotherapy. Procedure-related items may include surgery details, anesthesia details, blood transfusions, and administration. Test-related items may include pathology interpretation results, immunochemistry test results, molecular genetic test results, and electrocardiogram results. Patient observation items are clinical information related to the emergency department or intensive care unit, and may include emergency department encounters, intensive care unit encounters, medication infusions, sensorium assessments, clinical observations, and the like.

The analysis part may include items that contain derivative information necessary for studying and analyzing data from the items of the core part and the items of the extension part. For example, items classified into the analysis part may include treatment pathway, initial treatment, line of therapy, event information, and the like.

When a table specification of a new item to be included in the medical data library 100 is input, the apparatus 10 may determine whether the item is essential, whether the item is specialized, or the purpose of utilization, based on information about the new item, and classify the new item into any one of the core part, the expansion part, or the analysis part. Alternatively, the apparatus 10 may receive input from the user regarding where the new item should be located. The apparatus 10 may generate table information and a table structure for the new item using a structured query language (e.g., SQL) through a data definition language generation function. Based on the table information of the new item, the apparatus 10 may set the table generation order of the new item to ensure that the tables of the items used for generating a certain table of the new item are generated in advance. For example, the table generation order may be set such that the tables of the items in the core part and the expansion part are generated before the tables of the items in the analysis part. If there is a hierarchical relationship between the items, the generation order may be prioritized accordingly.

For example, the apparatus 10 may receive input of patient information items and a table specification as new items to be added in the medical data library 100. Since the patient information item is common data for all patients encountering the hospital, the apparatus 10 may classify the patient information item into the core part. The apparatus 10 may generate structured query language codes to manage the information contained in the table specification of the patient information items, such as age, gender, nationality, blood type, cause of death, and like that constitute the columns of the table. Since a patient identification number assigned in the patient information is used as a foreign key (FK) for all tables, the apparatus 10 may set the highest priority to the table of the patient information items, setting the generation order to 1.

For example, the apparatus 10 may receive input of a chemotherapy infusion protocol item and the table specification as new items to be added in the medical data library 100. Since the chemotherapy infusion protocol item is data generated when a patient diagnosed with a cancer receives chemotherapy treatment, the apparatus 10 may classify the chemotherapy infusion protocol item into the extension part. The apparatus 10 may generate structured query language codes to manage the information contained in the table specification of the chemotherapy infusion protocol item, such as the number of times of chemotherapy infusions, start date, end date, regimen, dosage, and the like that constitute the columns of the table. Since the table of the chemotherapy infusion protocol item uses the patient identification number, encounter number, and prescription number as foreign keys (FKs), the apparatus 10 may set the order of table generation to ensure that the table of the chemotherapy infusion protocol item is generated after the patient information table, encounter table, and prescription table.

For example, the apparatus 10 may receive input of a treatment pathway item and a table specification as new items to be added in the medical data library 100. Since the treatment pathway item is an item that integrates data from a medication order item and a surgery/procedure item in the core part, and a radio therapy item, a chemotherapy infusion protocol item, and a surgery details item in the extension part to an analysis code, to analyze a process for treating a disease, the apparatus 10 may classify the treatment pathway item into the analysis item. The apparatus 10 may generate structured query language codes to manage the information contained in the table specification of the treatment pathway item, such as treatment type, treatment start/end date, sequence, treatment result, and like that constitute the columns of the table. The apparatus 10 may set the order of table generation to ensure that the table of treatment pathway items is generated after the tables of the core part and the extension part.

In response to a request to generate a DB for study purposes of a specific disease, the apparatus 10 selects items for study purposes of the specific disease from the medical data library 100 and generates a table of the selected items. In this case, the apparatus 10 applies a fabric condition to extract a patient group specific to the disease to generate a table of each item using only data for the patient group of the specific disease.

The items in the medical data library 100 may be preset for selection based on the disease and study purposes. For example, a patient information item in the core part may be set to be selected by default when generating the DB for disease-specific study. The chemotherapy infusion protocol item in the extension part is set to be selected by default when generating the DB related to a cancer disease, but may be manually selected by the user when generating the DB related to other diseases. The treatment pathway item in the analysis part may be set to be selected by default when the purpose of the study is to analyze the treatment process.

The apparatus 10 may generate a table of patient information items using only data for the patient group of the specific disease by applying a fabric condition for extracting a patient group of a specific disease. The apparatus 10 may generate a table of the chemotherapy infusion protocol item using only data for a patient group of a cancer disease by applying a fabric condition for extracting the patient group of the cancer disease. The apparatus 10 may generate a table of a treatment pathway item using only data for a patient group of a specific disease by applying a fabric condition for extracting the patient group of the specific disease.

Referring to FIG. 2, the apparatus 10 may transform unstructured data 310 stored in the CDW 1 into a structured table 320 using an artificial intelligence (AI) model 300. For example, test results such as pathology interpretation result, immunochemistry test result, molecular genetics test result, and the like are stored in report formats. Therefore, the apparatus 10 may use the AI model 300, such as a large language model (LLM) or text recognition model, to extract values of columns (fields) of the table from the reports, and generate records in the table.

For example, the apparatus 10 may obtain immunochemistry test reports of a patient group with a specific disease by applying a fabric condition to extract the patient group with the specific disease. The apparatus 10 may generate a table of immunochemistry test result items using the AI model 300 by extracting information of table such as test type pathology number, diagnosis name, surgical pathology diagnosis name, biomarker, negative-positive code, test result content, test result symbolic character, test result numerical value, and test result unit, from the immunochemistry test report.

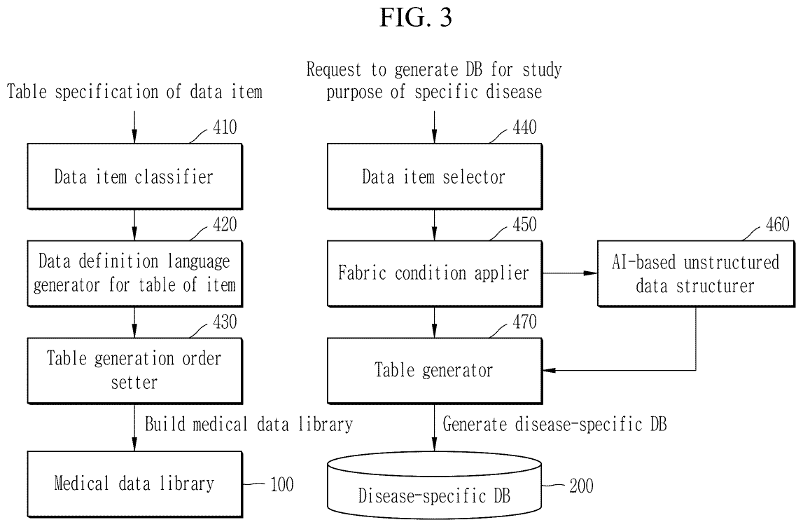

FIG. 3 is a diagram illustrating functions implemented in the apparatus for providing the data fabric-based disease-specific database according to the embodiment.

Referring to FIG. 3, the apparatus 10 may include a data item classifier 410, a data definition language generator 420, and a table generation order setter 430 for building the medical data library 100. Here, the data item classifier 410, the data definition language generator 420, and table generation order setter 430 are separated to illustrate representative operations of the apparatus 10.

When a table specification of data items to be included in the medical data library 100 (e.g., a patient information item, a chemotherapy infusion protocol item, and a treatment pathway item) is provided, the data item classifier 410 may determine whether the data items are essential, whether the data items are specialized, and utilization purposes based on information in the items, and may classify the corresponding item into any one of a core part, an extension part, or an analysis part. On the other hand, the items classified into the extension part may be further classified into the categories, such as disease, procedure, test result, continuous observation (simply, continuous obs), others, and each category may be further subdivided. In this case, the data item classifier 410 may classify the corresponding items into at least one category.

The data definition language generator 420 may generate the table information and structure for each item using a structured query language (e.g., SQL). Information may be extracted from the CDW 1 by the generated SQL code, and a table of the corresponding item may be generated from the extracted information. For the table generation, fabric conditions are applied to ensure that the table is generated using information extracted from the data from the patient group with the specific disease.

The table generation order setter 430 may set the sequence in which tables of corresponding items are generated based on the table information of each item, such that the table for the item used for generating a table of certain item are generated in advance. For example, the table generation order may be set such that the tables of the items in the core part and the extension part are generated before the tables of the items in the analysis part. If there is a hierarchical relationship between the items, the generation order may be prioritized accordingly.

The apparatus 10 may generate the disease-specific DB 200 using the construct medical data library 100, and to this end, the apparatus 10 may include a data item selector 440, a fabric condition applier 450, an AI-based unstructured data structurer 460, and a table generator 470. Here, the data item selector 440, the fabric condition applier 450, the AI-based unstructured data structurer 460, and the table generator 470 are separated to illustrate representative operations of the apparatus 10.

The data item selector 440 may select items from the medical data library 100 for study purposes for a specific disease in response to a request to generate a DB for study purposes for the specific disease. To this end, the data item selector 440 may set items to be selected by default from the medical data library 100 by disease and/or study purpose, and select items to make up the DB to meet the user request. For example, patient information items in the core part may be set to be selected by default for generation of any disease-specific database. The chemotherapy infusion protocol item is set to be selected by default when generating a database related to cancer diseases, but may be manually selected by the user when generating a database related to other diseases. The treatment pathway item may be set to be selected by default when the study purpose is to analyze the treatment process.

The fabric condition applier 450 may store fabric conditions that include medical conditions for extracting patient groups by disease and variables required for study purposes, and may apply fabric conditions for specific diseases to table generation.

The AI-based unstructured data structurer 460 may transform unstructured data stored in the CDW 1 into structured tables using the AI model 300.

The table generator 470 may use the fabric condition for a specific disease to restrict data extraction range to the data for a patient group with a specific disease, and generate tables of selected items using the data for the patient group of the specific disease. The table generator 470 may generate the tables by transforming the data for the patient group of the specific disease to be suitable to a table structure for each item. The table generator 470 may apply conversion rules to the target data to generate a desired table by using a data conversion code written in a structured query language. For example, the conversion rules may include rules to generate data for a patient group of a specific disease, exclude anomalies, exclude cancellation information, and use actual performed data. Further, the conversion rules may include rules to combine and generate tables for prescription, result, and codemaster into a single table for immediate use in performing analysis, to generate a single table with the same type of information, and to generate a table by structuring unstructured data using the AI model 300.

The disease-specific DB 200 may be built including the tables generated by the table generator 470.

The apparatus 10 may generate a table of patient information items using only data for a patient group of a specific disease by applying a fabric condition for extracting the patient group of the specific disease. The apparatus 10 may generate a table of chemotherapy infusion protocol items using only data for a patient group of a cancer disease by applying a fabric condition for extracting the patient group of the cancer disease. The apparatus 10 may generate a table of treatment pathway items using only data for a patient group of a specific disease by applying a fabric condition for extracting the patient group of the specific disease.

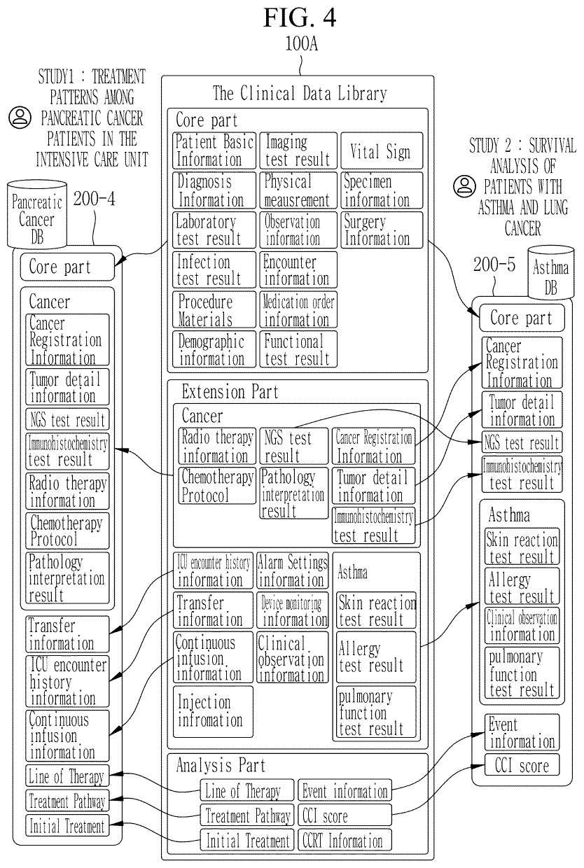

FIG. 4 is a diagram illustrating an example of generation of a disease-specific database according to the embodiment.

Referring to FIG. 4, the apparatus 10 may construct a medical data library 100A including selectable items based on a specific disease and study purpose. The medical data library 100A includes items classified into a core part, an extension part, and an analysis part, and a table of each item is generated using data for a patient group of a specific disease defined by a fabric condition. SQL codes are used to generate the tables from the CDW 1.

The items in the medical data library 100A may be selected based on a study purpose for a specific disease. For example, items in the core part may be automatically selected, items related to the specific disease and study purpose may be selected in the extension part, and items related to the study purpose may be selected in the analysis part.

For example, it is assumed that the apparatus 10 receives a request to generate a DB for “Study: Treatment Patterns Among Pancreatic Cancer Patients in the Intensive Care Unit”. The apparatus 10 may select all of the items in the core part of the medical data library 100A. Among the items in the extension part, the apparatus 10 may select items related to cancer diseases (e.g., cancer registration information, tumor detail information, next-generation sequencing (NGS) test result, immunohistochemistry test result, radio therapy information, chemotherapy protocol, and pathology interpretation result), intensive care unit-related items (e.g., transfer information, ICU encounter history information, and continuous infusion information). The apparatus 10 may select, from among the items in the analysis part, items related to the study of treatment patterns (e.g., Line of Therapy, Treatment Pathway, and initial treatment). When generating a table of each selected item, the apparatus 10 may apply a fabric condition to extract a patient group who have been treated in the ICU and another fabric condition to extract a patient group related to a pancreatic cancer, resulting in generating a table of each item using only data for pancreatic cancer patients who have admitted to the ICU. In this case, the apparatus 10 may verify the table generation order of the selected items and generate the tables of the items according to the table generation order. The apparatus 10 may complete a requested disease-specific DB 200-4 using the generated tables.

For example, it is assumed that the apparatus 10 receives a “Study: Survival Analysis of Patients with Asthma and Lung Cancer”. The apparatus 10 may select all of the items in the core part of the medical data library 100A. Among the items in the extension part, the apparatus 10 may select items related to the cancer disease (e.g., cancer registration information, tumor detail information, next-generation sequencing (NGS) test result, and Immunohistochemistry test result) and items related to the asthma disease (e.g., skin reaction test result, allergy test result, and pulmonary function test result). The apparatus 10 may select items related to a survival analysis study (e.g., event information, and CCI score) among the items in the analysis part. When generating a table of each selected item, the apparatus 10 may apply a fabric condition to extract the patient groups related to “lung cancer” and “asthma”, and generate a table of each item using only data for the patient groups of “lung cancer” and “asthma”. In this case, the apparatus 10 may verify the table generation order of the selected items and generate the tables of the items according to the table generation order. The apparatus 10 may complete a requested disease-specific DB 200-5 by using the generated tables.

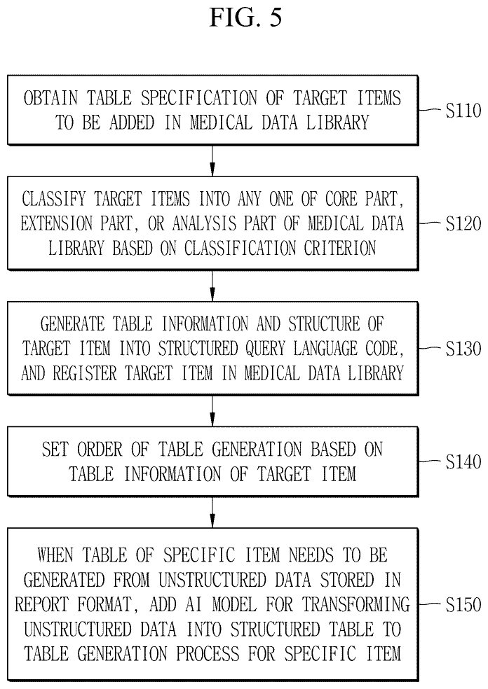

FIG. 5 is a flow diagram illustrating a method of building a medical data library according to an embodiment.

Referring to FIG. 5, the apparatus 10 obtains a table specification of target item to be added in the medical data library 100 (S110).

The apparatus 10 classifies the target item into any one of a core part, an extension part, or an analysis part of the medical data library 100 based on a classification criterion (S120). The classification criterion may include whether the target item is essential data for DB generation, whether the target item is specialized data, the purpose of utilization, and the like. When the target item is essential clinical information that is used as a default for studies, the target item may be classified into the core part. When the target item is clinical information that is optionally used based on the disease, applied condition, and study purpose, the target item may be classified into the extension part. When the target item includes derived information that is necessary for studying and analyzing the clinical information, the target item may be classified into the analysis part.

The apparatus 10 generates the table information and structure of the target item into a structured query language code, and registers the target item in the medical data library 100 (S130). Information may be extracted from the CDW 1 by the code generated in the structured query language, and a table record of the corresponding item may be generated by the extracted information.

The apparatus 10 sets an order of table generation based on the table information of the target item (S140). The table generation order may be determined based on the foreign keys (FKs) connected when the table of the target item is generated. For example, the table generation order may be set such that the tables of the items in the core part and the extension part is generated first, followed by the tables of the items in the analysis part. If there is a hierarchical relationship between the items, the generation order may be prioritized accordingly.

On the other hand, when the table of the specific item (e.g., immunochemistry test results) needs to be generated from unstructured data stored in a report format, the apparatus 10 adds the AI model 300 for transforming the unstructured data into a structured table to the table generation process for the specific item (S150). The AI model 300 may be implemented as an LLM, a text recognition model, or the like.

The items registered in the medical data library 100 may be provided in the selectable form for study of a specific disease, and implemented with a data fabric architecture in which tables of the selected items are generated using only data for patient groups according to disease-specific fabric conditions.

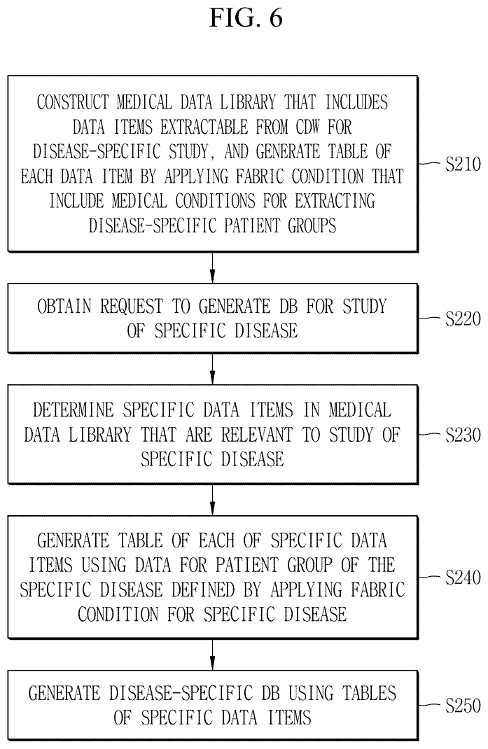

FIG. 6 is a flow diagram illustrating a method of generating a disease-specific database according to the embodiment.

Referring to FIG. 6, the apparatus 10 constructs a medical data library that includes data items extractable from the CDW 1 for disease-specific study, and generates a table of each data item by applying a fabric condition that include medical conditions for extracting disease-specific patient groups (S210).

The apparatus 10 obtains a request to generate a DB for study of a specific disease (S220). The apparatus 10 may obtain the request including a disease name and a data search period. The apparatus 10 may extract the number of patients with the specific disease from the CDW 1 and provided the extracted number of patients. The apparatus 10 may provide statistics of patient characteristics, such as age, gender, and nationality, to allow the user to confirm that the number of patients is appropriate for the study.

The apparatus 10 determines specific data items in the medical data library 100 that are relevant to the study of the specific disease (S230). The apparatus 10 may provide a user interface for a user to confirm the items required for the disease-specific DB. The apparatus 10 may manage the default items selected from the medical data library 100 by disease and/or study purpose, and may provide the user interface with the default items relevant to the request. The apparatus 10 may provide the user interface with selectable data items for study of the specific disease, and determine the data items confirmed in the user interface to be specific data items. Based on the roles of the items in the medical data library 100 and the relationships between the items, the apparatus 10 may recommend to the user the items required for the disease-specific DB to assist the user in selecting the items.

The apparatus 10 generates a table of each of the specific data items using data for the patient group of the specific disease defined by applying the fabric condition for the specific disease (S240). The fabric condition may further include variables that need to be set for the purpose of the study. The apparatus 10 may utilize the AI model 300 for transforming unstructured data into structured tables when generating the table of the specific item from unstructured data. The apparatus 10 may generate the table of the specific item by extracting a table record value from the unstructured data via the AI model 300. In this case, the apparatus 10 may determine the order of the table generation for the items, and may generate the tables according to the order.

The apparatus 10 generates a disease-specific DB using the tables of the specific data items (S250).

As such, according to the embodiments, databases for real-world evidence (RWE) analysis may be configured quickly and sophisticatedly.

According to the embodiments, study-specific databases that are built while relying on researcher's ability may be generated with consistent quality.

According to the embodiments, the data items within the medical data library may be freely combined to quickly generate disease-specific databases for study purposes.

According to the embodiments, data items in a medical data library may be used by multiple researchers to generate customized databases, and previously generated disease-specific databases may be reused.

The exemplary embodiments of the present disclosure described above are not only implemented through the apparatus and method, but may also be implemented through programs that realize functions corresponding to the configurations of the exemplary embodiment of the present disclosure, or through recording media on which the programs are recorded.

Although an exemplary embodiment of the present disclosure has been described in detail, the scope of the present disclosure is not limited by the exemplary embodiment. Various changes and modifications using the basic concept of the present disclosure defined in the accompanying claims by those skilled in the art shall be construed to belong to the scope of the present disclosure.

1. A method of an apparatus operating by at least one processor, the method comprising:
constructing a medical data library that includes data items extractable from a clinical data warehouse for disease-specific study, and generates a table of each data item by applying a disease-specific fabric condition that includes medical conditions for extracting a disease-specific patient group;
obtaining a request to generate a database for study of a specific disease;
determining, from the medical data library, specific data items relevant to the study of the specific disease;
generating a table of each of the specific data items using data for a patient group of the specific disease defined by applying a fabric condition of the specific disease; and
generating a database for the study of the specific disease using the tables of the specific data items.

2. The method of claim 1, wherein the determining the specific data items comprises:
providing a user interface with selectable data items for the study of the specific disease; and
determining data items confirmed in the user interface to the specific data items.

3. The method of claim 2, wherein the providing the user interface comprises
managing default items selected from the medical data library by disease or study purpose, and providing the user interface with default items related to the request to generate the database.

4. The method of claim 1, wherein the generating the table of each of the specific data items comprises,
when a table of a specific data item needs to be generated from unstructured data, extracting a table record value from the unstructured data using an artificial intelligence model to generate the table of the specific data item.

5. The method of claim 1, wherein the generating the table of each of the specific data items comprises:
determining an order of table generation of the specific data items; and
generating the table according to the order.

6. The method of claim 1, wherein
the data items in the medical data library are classified into any one of a core part, an extension part, or an analysis part,
the core part includes data items related to essential clinical information being used as a default for studies,
the extension part includes data items related to clinical information being optionally used based on a disease or study purpose, and
the analysis part includes data items related to derivative information necessary for studying and analyzing clinical information.

7. The method of claim 1, wherein the obtaining the request to generate the database comprises
receiving an input of a disease name of the specific disease.

8. The method of claim 1, further comprising:
after obtaining the request to generate the database, extracting a number of patients with the specific disease from the clinical data warehouse and providing the number of patients of the specific disease.

9. A method of an apparatus operating by at least one processor, the method comprising:
obtaining a table specification of target item to be added in a medical data library;
classifying the target item into any one of a core part, an extension part, or an analysis part of the medical data library based on a classification criterion; and
generating table information and structure of the target item into a structured query language code, and registering the target item in the medical data library,
wherein the medical data library is constructed to include data items extractable from a clinical data warehouse for disease-specific study, and to generate a table of each data item by applying a disease-specific fabric condition that includes medical conditions for extracting a disease-specific patient group.

10. The method of claim 9, wherein the classifying comprises:
when the target item is essential clinical information being used as a default for studies, classifying the target item into the core part;
when the target item is clinical information being optionally used for a disease or study purposes, classifying the target item into the extension part; and
when the target item is derivative information necessary for studying and analyzing clinical information, classifying the target item into the analysis part.

11. The method of claim 9, further comprising:
setting an order of table generation based on table information of the target item.

12. The method of claim 9, further comprising:
when a table for the target item needs to be generated from unstructured data, adding an artificial intelligence model to a table generation process for the target item to convert the unstructured data into a structured table.

13. The method of claim 9, further comprising:
obtaining a request to generate a database for study of a specific disease;
determining, from the medical data library, specific data items relevant to the study of the specific disease;
generating a table of each of the specific data items using data for a patient group of the specific disease defined by applying a fabric condition of the specific disease; and
generating a database for the study of the specific disease using the tables of the specific data items.

14. An apparatus comprising:
a memory; and
a processor for executing instructions stored in the memory,
wherein the processor is configured to, by executing the instructions,
construct a medical data library to include data items extractable from a clinical data warehouse for disease-specific study, and to generate a table of each data item by applying a disease-specific fabric condition that includes medical conditions for extracting a disease-specific patient group,
generate a table of each of the data items selected in the medical data library using data for a patient group of a specific disease defined by applying a fabric condition of the specific disease, and provide a database including the generated tables for study of the specific disease.

15. The apparatus of claim 14, wherein the processor is configured to
obtain a request to generate a database for study of the specific disease,
determine, from the medical data library, specific data items relevant to the study of the specific disease,
generate a table of each of the specific data items using data for a patient group of the specific disease defined by applying a fabric condition of the specific disease, and
generate a database for the study of the specific disease using the tables of the specific data items.

16. The apparatus of claim 14, wherein the processor is configured to
provide a user interface with selectable data items in the medical data library for the study of the specific disease, and
determine data items confirmed in the user interface to the specific data items.

17. The apparatus of claim 16, wherein the processor is configured to
manage default items selected from the medical data library by disease or study purpose, and provide the user interface with default items related to the request to generate the database.

18. The apparatus of claim 14, wherein the processor is configured to,
when a table of a specific data item needs to be generated from unstructured data, extract a table record value from the unstructured data using an artificial intelligence model to generate the table of the specific data item.

19. The apparatus of claim 14, wherein the processor is configured to
determine an order of table generation of the specific data items, and generate the table according to the order.

20. The apparatus of claim 14, wherein the data items in the medical data library are classified into any one of a core part, an extension part, or an analysis part,
the core part includes data items related to essential clinical information being used as a default for studies,
the extension part includes data items related to clinical information being optionally used based on a disease or study purpose, and
the analysis part includes data items related to derivative information necessary for studying and analyzing clinical information.
